(19) United States
(12) Patent Application Publication
(10) Pub. No.: US 2025/0176937 A1
(43) Pub. Date: Jun. 5, 2025

(54) ULTRASONIC DEVICE, HEAD HOLDER, AND METHOD FOR PROCESSING ULTRASONIC SIGNALS

(71) Applicant: NATIONAL INSTITUTE OF ADVANCED INDUSTRIAL SCIENCE AND TECHNOLOGY, Tokyo (JP)

(72) Inventors: Keigo HIKISHIMA, Tsukuba-shi, Ibaraki (JP); Naotaka NITTA, Tsukuba-shi, Ibaraki (JP); Kiyoshi YOSHINAKA, Tsukuba-shi, Ibaraki (JP); Ryosuke TSUMURA, Tsukuba-shi, Ibaraki (JP); Naoyuki MIYASAKA, Tsukuba-shi, Ibaraki (JP)

(21) Appl. No.: 18/685,940

(22) PCT Filed: Aug. 24, 2022
(86) PCT No.: PCT/JP2022/031786

§ 371 Date: Feb. 23, 2024

(30) Foreign Application Priority Data
Aug. 27, 2021 (JP) 2021-139000

(51) Int. Cl. A61B 8/00; A61B 8/06

(52) U.S. Cl. CPC A61B 8/4477; A61B 8/06; A61B 8/4218; A61B 8/463; A61B 8/466; A61B 8/483; A61B 8/54; A61B 2503/04

(57) ABSTRACT

The present invention discloses a technology for measuring brain function suitable for an infant or a moving subject who cannot be controlled easily during measurement. One embodiment of the present disclosure pertains to an ultrasound device that comprises: multiple ultrasound probes that receive/transmit ultrasound signals from/to multiple brain areas via multiple head regions and are disposed corresponding to the respective head regions; a control unit that controls the respective ultrasound probes; and an image processing unit that generates brain function network information calculated from blood flow states among the respective brain areas based on measurement results acquired from the respective ultrasound probes.

### TECHNICAL FIELD

The present disclosure relates to an ultrasound device, a head holder, and an ultrasound signal processing method.

### BACKGROUND ART

The human brain forms the center of the nervous system and performs various functions such as movement control, sensation, autonomic control, language, emotion, and cognition. It is known that these functions can be realized by cooperation of a plurality of brain regions in the brain. For this reason, brain functional network information indicating an interregional connection state of neural activity in the brain is available for brain diagnosis or the like.

Several measurement methods for such brain functional network information have been proposed. Examples thereof include scalp electroencephalography, magnetoencephalography, optical topography, brain functional magnetic resonance imaging, and brain functional ultrasound measurement. The scalp electroencephalography is a technique to measure potential changes in the brain in unit of millisecond using electrodes placed on the scalp, and mainly analyzes the activity in the cortical region. The magnetoencephalography is a technique to measure changes of magnetic field associated with electrical activity of the brain using a superconducting quantum interferometer in unit of millisecond, and to analyze the brain activity mainly in the cortical region. The optical topography is a technique to measure cerebral hemodynamics associated with neural activity by transmitting and receiving near-infrared rays using optical fibers placed on the scalp, and mainly analyzes the brain activity in the cortical region. The brain functional magnetic resonance imaging is a technique to measure cerebral hemodynamics associated with neural activity using a magnetic resonance imaging apparatus, and to analyze the brain activity in an entire brain region. The brain functional ultrasound measurement is a technique to measure cerebral hemodynamics associated with neural activity by Doppler ultrasound measurement, and to analyze the brain activity.

### CITATION LIST

### Patent Literature

### PTL 1

Japanese Patent Application Laid-Open No. 2003-79626

### PTL 2

Japanese Unexamined Patent Application Publication (Translation of PCT Application) No. 2017-524430

### PTL 3

U.S. Patent Application Publication No. 2018/0177487

### Non-Patent Literature

### NPL 1

Baranger J, Demene C, Frerot A, Faure F, Delanoë C, Serroune H, Houdouin A, Mairesse J, Biran V, Baud O, Tanter M. Bedside functional monitoring of the dynamic brain connectivity in human neonates. Nat Commun. 2021 Feb. 17; 12(1):1080. doi: 10.1038/s41467-021-21387-x.

### NPL 2

Demene C, Pernot M, Biran V, Alison M, Fink M, Baud O, Tanter M. Ultrafast Doppler reveals the mapping of cerebral vascular resistivity in neonates. J Cereb Blood Flow Metab. 2014 June; 34(6):1009-17. doi: 10.1038/jcbfm.2014.49.

### NPL 3

Demene C, Baranger J, Bernal M, Delanoe C, Auvin S, Biran V, Alison M, Mairesse J, Harribaud E, Pernot M, Tanter M, Baud O. Functional ultrasound imaging of brain activity in human newborns. Sci Transl Med. 2017 Oct. 11; 9(411):eaah6756. doi: 10.1126/scitranslmed.aah6756.

### SUMMARY OF INVENTION

### Technical Problem

However, there are some problems in cases where these measurement methods are used for subjects such as infants (defined herein as infants up to 1 year old) who are unlikely to stay still during measurement, and to control their posture and the like, and for subjects who are moving.

For example, the scalp electroencephalography can directly capture the electrical activity of the brain in unit of millisecond, but is limited to signal measurement chiefly at a depth of about 10 mm from the surface of the brain. In addition, in measurement of infants, artifacts such as body motion and crying are likely to be mixed, and it is often difficult to attach and maintain electrodes. Thus, measurement during natural sleep and/or use of a sedative are often required.

Further, the magnetoencephalography can capture the electrical activity of the brain in unit of millisecond as in the electroencephalography, but is poor at measurement at the deep part of the brain, and a measurement apparatus is large and expensive. In addition, similar to the scalp electroencephalography, artifacts such as body motion and crying are likely to be mixed in measurement for infants, and measurement during natural sleep and/or use of a sedative are often required.

The optical topography is limited to signal measurement at a depth of about 20 mm from the scalp, although body motion has a relatively small effect. The optical topography contains external information other than information on the brain.

The brain functional magnetic resonance imaging can capture the activity of the whole brain, but a measurement apparatus is large and expensive. In addition, artifacts such as body motion and crying are likely to be mixed in measurement for infants, and measurement during natural sleep and/or use of a sedative are often required. Furthermore, acoustic noises caused during the brain functional magnetic resonance imaging reduce the connectivity of a brain network appearing while a subject is in the resting state, thus affecting the accurate evaluation of the resting brain network.

The brain functional ultrasound measurement is comparatively less effected by the body motion. However, ultrasound waves are significantly attenuated and reflected in a skull. Thus, the brain functional ultrasound measurement is limited to observation of partial brain regions in which the fontanelles for infants, which are a connective tissue membrane where the skull has not been ossified, or the temporal bone window for adults where the skull is thin are used as acoustic windows.

In view of the above problems, an object of the present disclosure is to provide a technique for measuring a brain function, which is suitable for an infant who is difficult to control during measurement and a subject who is moving.

### Solution to Problem

One aspect of the present disclosure relates to an ultrasound device including: a plurality of ultrasound probes that transmit and receive an ultrasound wave to and from a plurality of brain regions via a plurality of head regions, the plurality of ultrasound probes being disposed corresponding to the respective head regions; a head holder including the plurality of ultrasound probes corresponding to the respective head regions; a controller that controls the plurality of ultrasound probes; and an image processor that generates brain functional network information calculated from a blood flow state of a blood flow among the plurality of brain regions based on a measurement result acquired from the plurality of ultrasound probes.

Another aspect of the present disclosure relates to a head holder including: a plurality of ultrasound probes that transmit and receive ultrasound waves to and from a plurality of brain regions via a plurality of head regions, wherein the plurality of ultrasound probes are disposed corresponding to the respective head regions.

Another aspect of the present disclosure relates to an ultrasound signal processing method executed by a computer, the ultrasound signal processing method including: acquiring a measurement result from a plurality of ultrasound probes that transmit and receive ultrasound waves to and from a plurality of brain regions via a plurality of head regions; and generating brain functional network information calculated from a blood flow state among the plurality of brain regions based on the measurement result, wherein the plurality of ultrasound probes are disposed corresponding to the respective head regions.

### Advantageous Effects of Invention

According to the present disclosure, it is possible to provide a technique for measuring a brain function, which is suitable for an infant who is difficult to control during measurement or a moving subject.

### BRIEF DESCRIPTION OF DRAWINGS

FIG. 1 schematically illustrates an ultrasound device according to one exemplary embodiment of the present disclosure;

FIG. 2 illustrates a two-dimensional array of transducers of an ultrasound probe according to one exemplary embodiment of the present disclosure;

FIG. 3 illustrates a head of an infant according to one exemplary embodiment of the present disclosure;

FIGS. 4A and 4B illustrate a head holder according to one exemplary embodiment of the present disclosure;

FIG. 5 illustrates a probe holder of the head holder according to one exemplary embodiment of the present disclosure;

FIGS. 6A and 6B illustrate examples of propagation of an ultrasound wave from the ultrasound probe according to one exemplary embodiment of the present disclosure;

FIG. 7 illustrates an emission pattern from a plurality of ultrasound probes according to one exemplary embodiment of the present disclosure;

FIG. 8 illustrates one exemplary hemodynamic response due to neuroexcitatory action;

FIGS. 9A and 9B illustrate local brain images measured by the ultrasound probes according to one exemplary embodiment of the present disclosure;

FIGS. 10A-C illustrate exemplary combination of images measured by the ultrasound probes according to one exemplary embodiment of the present disclosure;

FIG. 11 schematically illustrates an example of coordinate transformation to a three-dimensional brain atlas according to one exemplary embodiment of the present disclosure;

FIGS. 12A and 12B schematically illustrate correlation analyses between brain regions according to one exemplary embodiment of the present disclosure;

FIGS. 13A and 13B illustrate matrices representing correlations between the brain regions according to one exemplary embodiment of the present disclosure;

FIG. 14 illustrates a shortest path length and a clustering coefficient in a brain functional network according to one exemplary embodiment of the present disclosure;

FIG. 15 schematically illustrates an ultrasound device according to another exemplary embodiment of the present disclosure;

FIG. 16 illustrates a brain network abnormality of a developmental disorder model by ultrasound brain functional network measurement according to one exemplary embodiment of the present disclosure; and

FIG. 17 is a flowchart illustrating ultrasound signal processing according to one exemplary embodiment of the present disclosure.

### DESCRIPTION OF EMBODIMENTS

Hereinafter, embodiments of the present disclosure is described with reference to the drawings.

In the following exemplary embodiment, an ultrasound device is disclosed that utilize automatic tracking using a head holder or robotic arms provided with a plurality of ultrasound probes corresponding to a plurality of head regions to analyze interactions between a plurality of brain regions of a subject.

An ultrasound device according to one exemplary embodiment of the present disclosure is suitable for infants, for example, and the head holder includes a plurality of ultrasound probes disposed corresponding to respective positions of a plurality of openings in a head peculiar to the infants, that is, a plurality of fontanelles such as an anterior fontanelle and a posterior fontanelle. The ultrasound device uses the plurality of openings as acoustic windows of ultrasound waves to generate local brain images measured by the ultrasound probes based on ultrasound pulses transmitted and received to and from a plurality of brain regions of an infant and form a wide-area brain image of the entire brain from the generated local brain images. Then, the ultrasound device performs coordinate transformation on the wide-area brain image into a three-dimensional brain atlas and generates brain functional network information indicating the blood flow states of blood flows between the brain regions based on correlation of blood flow state between the brain regions in the three-dimensional brain atlas.

Thus, by transmitting the ultrasound pulses through the plurality of distant acoustic windows one after another for measurement, it is possible to acquire the brain functional network information on the brain functional network between the brain regions while suppressing a local temperature rise of the brain tissue due to a thermal effect of the ultrasound waves. In conventional transcranial ultrasound measurement methods, a single hand-held type applied to a fontanelle or a temporal bone window to image the brain. On the other hand, according to exemplary embodiments of the present disclosure, measurement and analysis based on the alternate control of a plurality of ultrasound probes via a plurality of head regions are utilized, and it is thus possible to automatically achieve observation of a wide area of the brain such as the cerebrum and the cerebellum for infants. Further, by fixing the ultrasound probes to probe holders disposed on the head holder such as a helmet or to the robotic arms capable of automatically tracking a head based on a head-shaped 4D image by a stereo-camera, it becomes possible to suppress an influence of the body motion of the subject. Thus, without measurement during natural sleep and/or use of a sedative for use in other non-invasive measurement methods for infants, it is possible to easily and safely conduct the measurement. Thus, according to the present disclosure, it is possible to provide a subject with a wide range of approaches for functional network evaluation in which a wide brain area is grasped integrally.

### [Ultrasound Device]

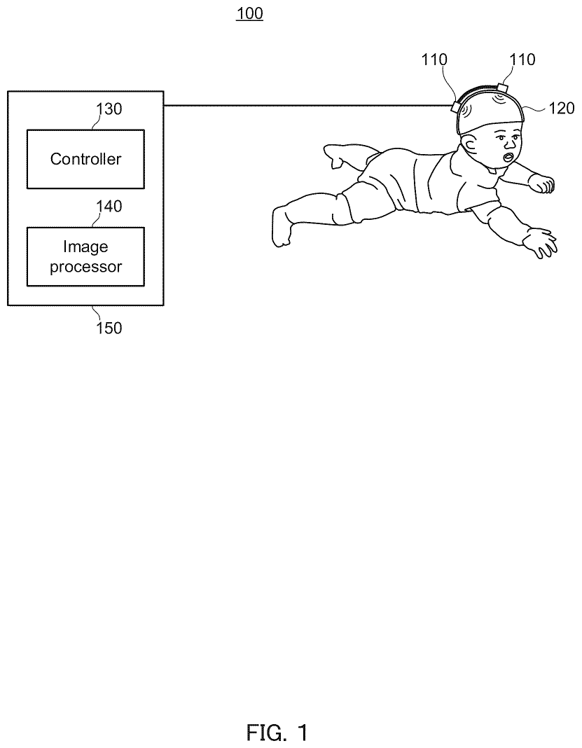

To begin with, an ultrasound device 100 according to one exemplary embodiment of the present disclosure is described with reference to FIGS. 1 to 16. The ultrasound device 100 according to the present exemplary embodiment is preferably used to measure the brains of infants. However, the ultrasound device according to the present disclosure is not limited to the use for the infants, and may be used for brain measurement for children, adults, elderly persons, or any other type of subject regardless of sex. Although infants are defined herein as being younger than one year of age, the ultrasound device according to the present disclosure is not limited to subjects of this age.

FIG. 1 schematically illustrates a configuration of the ultrasound device 100 according to one exemplary embodiment of the present disclosure. As illustrated in FIG. 1, the ultrasound device 100 includes a plurality of ultrasound probes 110, a head holder 120, a controller 130, and an image processor 140.

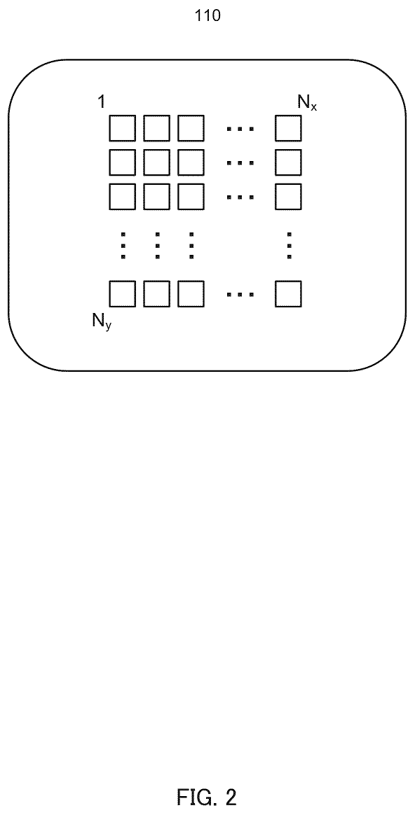

The plurality of ultrasound probes 110 transmit and receive ultrasound waves to and from a plurality of brain regions via a plurality of head regions. Specifically, each of the ultrasound probes 110 includes a transducer (piezoelectric element) that transmits an ultrasound signal toward an object and receives a reflected signal from the object. For example, the ultrasound probe 110 includes a plurality of transducers arranged in an array, transmits an ultrasound signal from each transducer to an object, and forwards to the controller 130 a measurement result obtained by receiving a reflected signal from the object. For example, as illustrated in FIG. 2, each of the ultrasound probes 110 may include two-dimensionally arranged (Nₓ×N_{y}) transducers composed of Nₓ transducers in the horizontal direction and N_{y} transducers in the vertical direction.

As illustrated in FIG. 1, the ultrasound probe 110 may use a spherical diffusion wave, which is a transmission wave spreading with distance, to observe the inside of the brain in a wide area and in a short time using a narrow region of the fontanelle as an acoustic window. In order to form the spherical diffusion wave, a virtual point sound source is disposed behind the two-dimensional array of transducers in an emission direction of the ultrasound wave. A difference in the arrival time of the ultrasound wave from the virtual point sound source to the transducers is set as delay time, and the controller 130 transmits drive signals to the respective transducers of the ultrasound probe 110. The controller 130 and the image processor 140 together constitute the control device 150 of the ultrasound probe 110, and the control device 150 and the ultrasound probe 110 may be connected to each other by a wire as illustrated in FIG. 1, or may be connected to each other by wireless.

Note that the shape of the ultrasound probe 110 may be any of a sector type, a convex type, a linear convex type, and a phased array type. In the example illustrated in FIG. 1, each of the ultrasound probes 110 emits the spherical diffusion wave, but the ultrasound probe 110 of the present disclosure is not limited thereto. The ultrasound probes 110 may have any suitable mechanism for forming a wavefront having an arbitrary shape, and may transmit an ultrasound signal of an arbitrary wavefront shape using the mechanism. For example, two-dimensional or three-dimensional plane waves or diffusion waves may be emitted.

The head holder 120 includes a plurality of ultrasound probes 110 corresponding to the plurality of head regions. For example, the head holder 120 may be a set of holders such as helmets, caps, or hoods having sizes of the lower limit of the head circumference of 33 cm for neonates to the upper limit of 60 cm for adults to cover the heads of subjects. Specifically, the head holder 120 may be realized as a helmet having a two-layer structure formed of a hard resin on the outside and a soft sponge on the inside. A soft sponge material can be used for the inside of the helmet to protect the head while the helmet is worn.

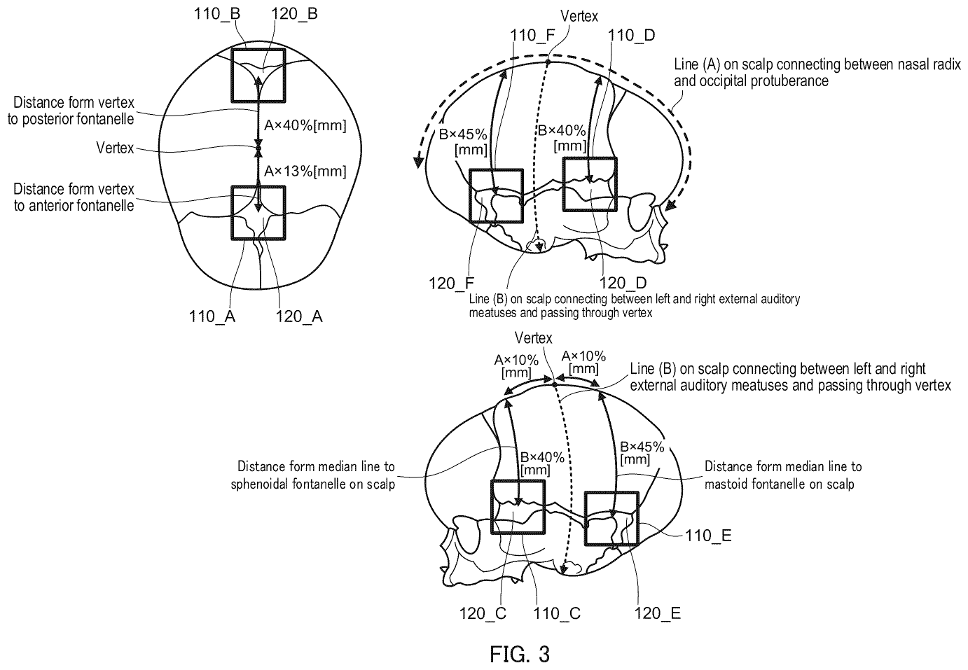

The skull of an infant has an opening (gap) in the skull called the fontanelles. Specifically, as illustrated in FIG. 3, the skull has six fontanelles: an anterior fontanelle 120_A, a posterior fontanelle 120_B, left and right sphenoidal fontanelles 120_C and 120_D, and left and right mastoid fontanelles 120_E and 120_F. In addition, the skull has a temporal bone window, which is a thin portion of the temporal bone, through which ultrasound waves may be easily transmitted even after growth. The ultrasound device 100 can use these head regions as acoustic windows for the ultrasound probes 110_A to 110_F to measure the brain of the subject by transmitting and receiving ultrasound signals to and from the brain. In the present embodiment, the head holder 120 is formed to hold the ultrasound probes 110_A to 110_F at positions corresponding to the six fontanelles, respectively. However, the head holder 120 according to the present disclosure is not limited thereto. The head holder 120 may hold three or more, four or more, five or more, or six ultrasound probes 110 for two or more head regions from among the anterior fontanelle, the posterior fontanelle, the left sphenoidal fontanelle, the right sphenoidal fontanelle, the left mastoid fontanelle, the right mastoid fontanelle and the temporal bone window. On the other hand, in order to measure the entire brain of the subject, it is desirable to provide the ultrasound probes 110 for as many head regions as possible.

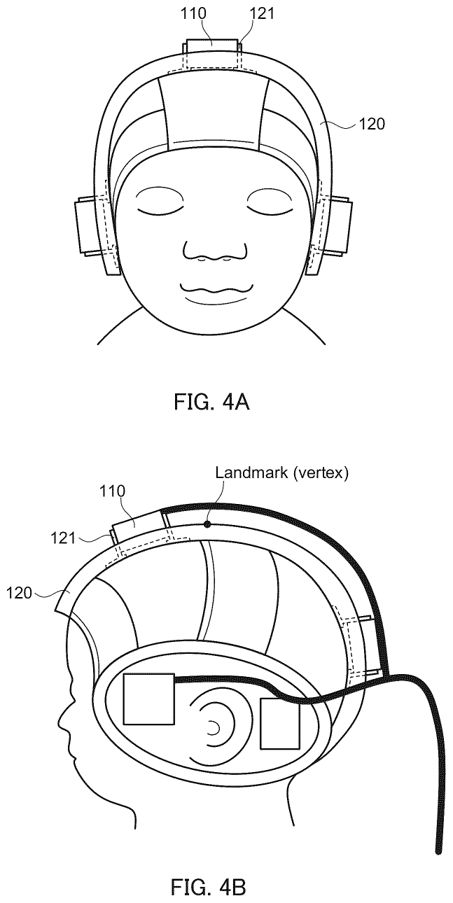

As illustrated in FIG. 4, each ultrasound probe 110 may be connected to a probe holder 121 fixed to the head holder 120 such as a helmet. Coupling to the probe holder 121 fixes the coordinates and tilt of the ultrasound probe 110 and helps to accurately construct a wide-area brain image of the entire brain from local brain images generated by ultrasound probes 110.

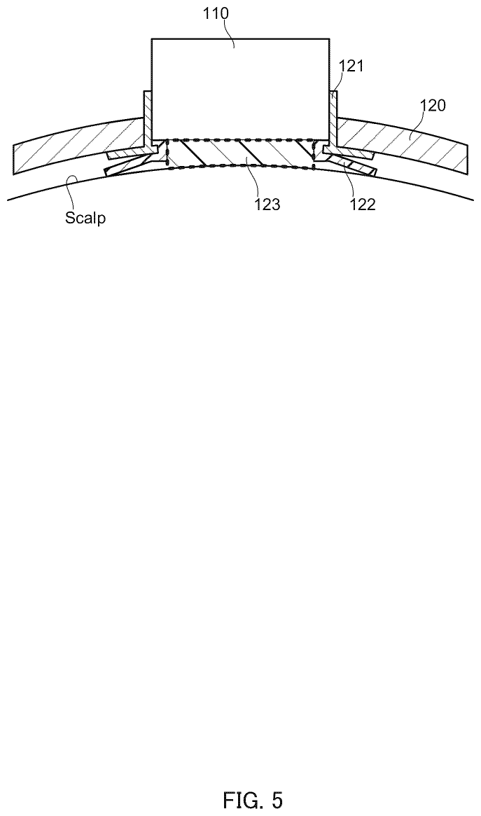

For example, each ultrasound probe 110 may be held by the probe holder 121 placed on the head holder 120, as illustrated in FIG. 5. In the illustrated example, the probe holder 121 is attached to the head holder 120 at a position corresponding to the fontanelle. In addition, a stopper is disposed inside the probe holder 121 so that the ultrasound probe 110 can avoid contacting with the head of the subject. Although the shape of the head varies between individuals, the individual variations can be largely absorbed by the combination of the head holder 120 and the probe holder 121. However, an adhesive silicon pad 122 may be attached between the probe holder 121 and the head such that the probe holder 121 fits securely to the scalp. The inside of the silicon pad 122 is filled with an ultrasound gel 123 that is sterilized and warmed to a body temperature, such that the acoustic impedance at a gap between the ultrasound probe 110 and the scalp is matched.

Several types of the head holder 120 and the probe holder 121 may be prepared so as to correspond to variations in the head size and shape of the subject and the positions of the fontanelles and temporal bone window. In addition, the head holder 120 may be formed so that the probe holders 121 can be placed at various positions on the head and can be adjusted to various head sizes. It is thus possible to perform safe measurement on the head of the subject while suppressing movement of the ultrasound probes 110 caused by body motion of the subject. Note that when the head holder 120 does not match the shape of the subject's head, the ultrasound probes 110 may be fixed by a rubber cap or the like. The cables connected to the ultrasound probes 110 may be bundled together and connected to probe sockets, as illustrated in FIG. 4.

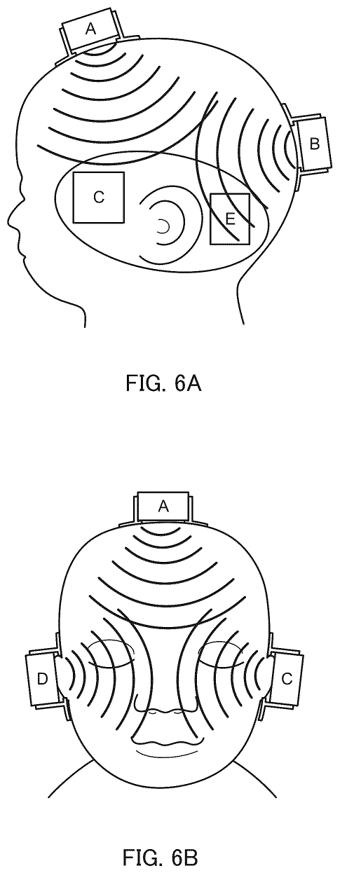

The controller 130 controls the plurality of the ultrasound probes 110. Specifically, the controller 130 controls the operation of the ultrasound probes 110 for ultrasound measurement on objects such as one or more brain regions in the brain, and receives measurement results from the ultrasound probes 110. Specifically, the controller 130 may control the driving of the plurality of ultrasound probes 110 such that transmission and reception of ultrasound signals by the ultrasound probes 110 do not interfere with one another. For example, as illustrated in FIG. 6, the ultrasound probes 110_A to 110_F may be disposed at the anterior fontanelle 120_A, the posterior fontanelle 120_B, the left sphenoidal fontanelle 120_C, the right sphenoidal fontanelle 120_D, the left mastoid fontanelle 120_E, the right mastoid fontanelle 120_F, respectively, and transmit and receive ultrasound signals by using these openings as acoustic windows.

For neonate's heads, the position of each ultrasound probe is defined as described below. Reference is made to the line (A) on the scalp connecting between the nasal radix and the occipital protuberance and the line (B) on the scalp connecting between the left and right external auditory meatuses and passing through the vertex in FIG. 3. The center of the ultrasound probe corresponding to the anterior fontanelle is placed at a position (serving as a center) anterior by 13% of the length of the line A from the midpoint (vertex) of the line A within ±5% of the length of the line A along the median line and within ±5% of the length of the line B along a line perpendicular to the line A. Also, the ultrasound probe corresponding to the posterior fontanelle is placed at a position (serving as a center) posterior from the vertex by 40% of the length of the line A within ±5% of the length of the line A along the median line and within ±5% of the length of the line B along a line perpendicular to the line A. In addition, the centers of the ultrasound probes corresponding to the left and right sphenoidal fontanelles are placed on a line on the scalp that is parallel to the line B and passes through a position anterior from the vertex by 10% of the length of the line A. The centers of the ultrasound probes corresponding to the left and right sphenoidal fontanelles are placed respectively at positions (serving as centers) on the opposite sides by 40% of the length of the line B from the intersection of the line B with the line A within ±5% of the length of the line B along of the parallel line and within ±5% of the length of the line A along lines perpendicular to the line B. Likewise, the centers of the ultrasound probes corresponding to the left and right mastoid fontanelles are placed on a line on the scalp that is parallel to the line B and passes through a position posterior from the vertex by 10% of the length of the line A. The centers of the ultrasound probes corresponding to the left and right mastoid fontanelles are placed respectively at positions (serving as centers) on the opposite sides by 45% of the length of the line B from the intersection of the line B with the line A within ±5% of the length of the line B along of the parallel line and within ±5% of the length of the line A along lines perpendicular to the line B. The head holder is provided with a landmark of the above-described vertex and the probe holders corresponding to the above-described ultrasound probe coordinates, and the ultrasound probes automatically correspond to the respective positions of the fontanelles by aligning a helmet landmark with the vertex.

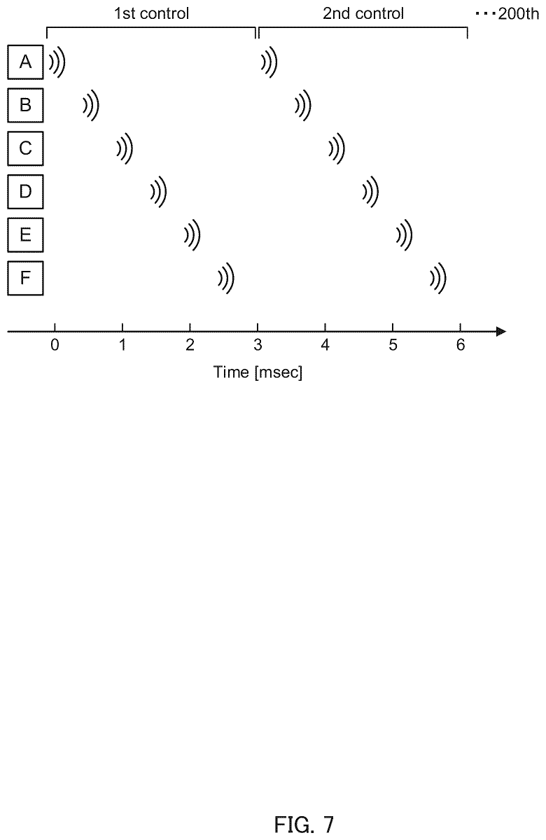

In one exemplary embodiment, the controller 130 may control the plurality of ultrasound probes 110 to transmit and receive ultrasound waves alternately. Specifically, the controller 130 may drive the ultrasound probes 110_A to 110_F in accordance with an emission pattern of ultrasound signals from the ultrasound probes 110_A to 110_F as illustrated in FIG. 7.

In the illustrated example, the ultrasound probe 110_A first performs ultrasound measurement by transmitting and receiving an ultrasound signal to and from the brain via the anterior fontanelle 120_A for a predetermined period of time, and transmits a measurement result to the controller 130.

Next, the ultrasound probe 110_B performs ultrasound measurement by transmitting and receiving an ultrasound signal to and from the brain via the posterior fontanelle 120_B for a predetermined period of time, and transmits a measurement result to the controller 130.

Next, the ultrasound probe 110_C performs ultrasound measurement by transmitting and receiving ultrasound signals to and from the brain via the left sphenoidal fontanelle 120_C for a predetermined period of time, and transmits a measurement result to the controller 130.

Next, the ultrasound probe 110_D performs ultrasound measurement by transmitting and receiving an ultrasound signal to and from the brain via the right sphenoidal fontanelle 120_D for a predetermined period of time, and transmits a measurement result to the controller 130.

Next, the ultrasound probe 110_E performs ultrasound measurement by transmitting and receiving an ultrasound signal to and from the brain via the left mastoid fontanelle 120_E for a predetermined period of time, and transmits a measurement result to the controller 130.

Next, the ultrasound probe 110_F performs ultrasound measurement by transmitting and receiving an ultrasound signal to and from the inside of the brain via the right mastoid fontanelle 120_F for a predetermined period of time, and transmits a measurement result to the controller 130.

When all the six ultrasound probes 110_A to 110_F complete the ultrasound measurement (Nₚ=6) in this manner, the ultrasound measurement by the ultrasound probes 110_A to 110_F as stated above is further repeated as illustrated in FIG. 7. In the illustrated example, the transmission and reception pattern for the ultrasound signals by the above-described ultrasound probes 110_A to 110_F is repeated 200 times (Nᵣ=200). As described above, by alternately transmitting the ultrasound pulses through the plurality of distant acoustic windows for measurement, it is possible to acquire brain functional network information on the brain functional network between the brain regions while suppressing a local temperature rise of the brain tissue due to a thermal effect of the ultrasound waves.

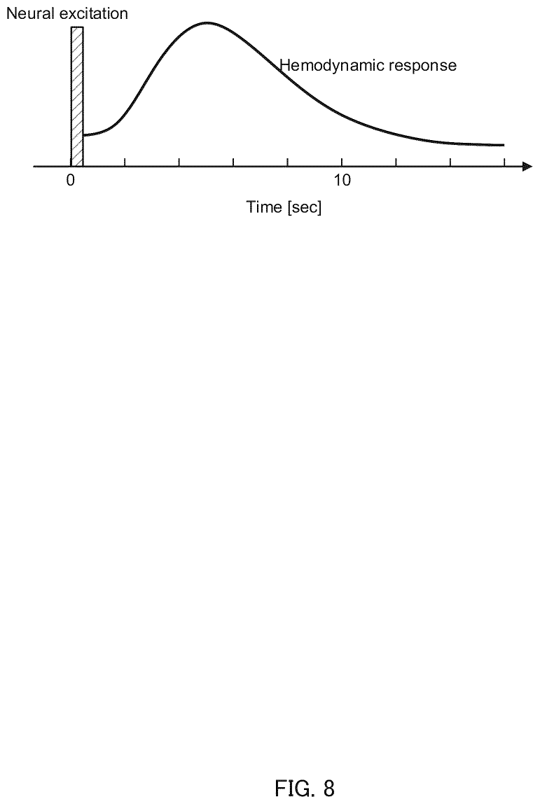

FIG. 8 illustrates changes in blood flow rate over time due to neuroexcitatory action according to an example. As illustrated in FIG. 8, it is known that when the neuroexcitatory action occurs, a change in blood flow state in the brain caused by the neuroexcitatory action appears as a hemodynamic response with a time lag of several seconds. Therefore, the number of repetitions of the transmission and reception pattern for the ultrasound signals may be set in accordance with a period during which the hemodynamic response can be observed.

However, the above-described transmission and reception pattern for the ultrasound signals is merely an example, and the transmission and reception pattern for the ultrasound signals by the ultrasound probes 110_A to 110_F according to the present disclosure is not limited thereto. In addition, ultrasound signals having the same wavefront shape do not need to be transmitted for all repetitions, and ultrasound signals having different wavefront shapes may be transmitted for the respective repetitions. Further, the ultrasound signals having the same wavefront shape do not need to be transmitted to all the ultrasound probes 110_A to 110_F, and the ultrasound signals having different wavefront shapes may be transmitted respectively to the ultrasound probes 110_A to 110_F.

When a reflected wave is received as a measurement result from each of the ultrasound probes 110_A to 110_F, the controller 130 passes the acquired measurement result to the image processor 140.

The image processor 140 generates the brain functional network information calculated from the blood flow states between the plurality of brain regions based on the measurement results acquired from the plurality of ultrasound probes 110_A to 110_F. Specifically, by using the measurement results acquired from the ultrasound probes 110_A to 110_F, the image processor 140 generates local brain images indicating one or more brain regions that can be measured by the ultrasound probes 110_A to 110_F based on the reflected waves of the transmitted ultrasound signals.

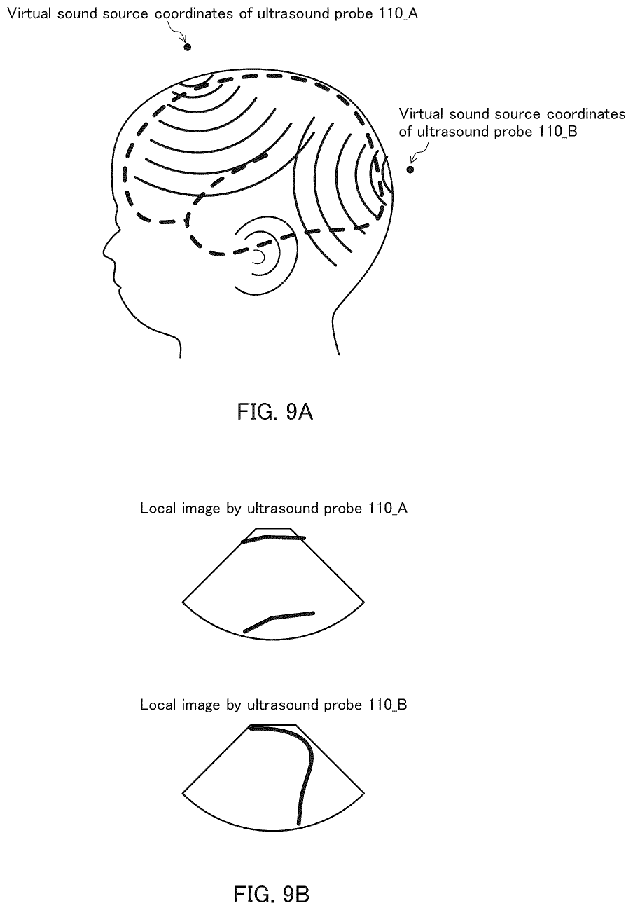

For example, in the example illustrated in FIG. 9, the ultrasound probe 110_A disposed at the anterior fontanelle 120_A is set to have, for example, the virtual sound source coordinates as illustrated and acquires a local brain image of a brain region within the reachable range of the ultrasound signal from the ultrasound probe 110_A as illustrated. The ultrasound probe 110_B disposed at the posterior fontanelle 120_B is set to have, for example, the virtual sound source coordinates as illustrated in the figure, and acquires a local brain image of a brain region within the reachable range of the ultrasound signal from the ultrasound probe 110_B as illustrated in the figure.

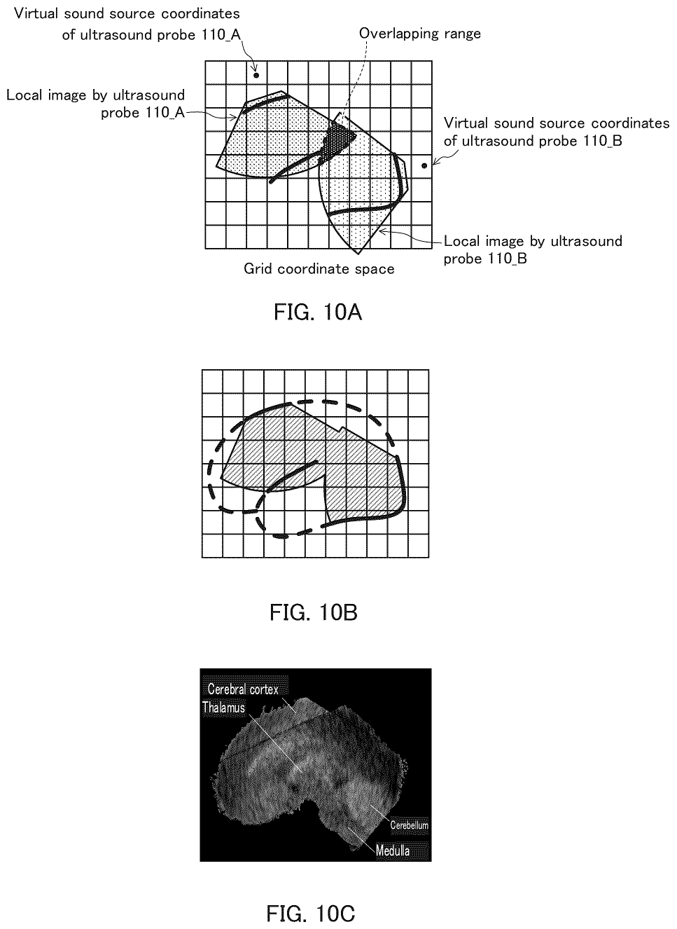

Upon generating the local brain images of the ultrasound probes 110_A to 110_F, the image processor 140 combines these local brain images to generate an image of the entire brain. For example, the image processor 140 may identify overlapping portions of the two local brain images as illustrated in FIG. 10A, and combine the two local brain images by superimposing the overlapping portions on each other. Likewise, the image processor 140 combines the local brain images by superimposing on each other the overlapping portions of the local brain images of the ultrasound probes 110_A to 110_F, so as to generate a wide-area brain image of the entire brain as illustrated in FIG. 10B. For example, the image processor 140 may reconstruct pieces of measurement data in multiple directions to acquire a brain wide-area ultrasound image as illustrated in FIG. 10C.

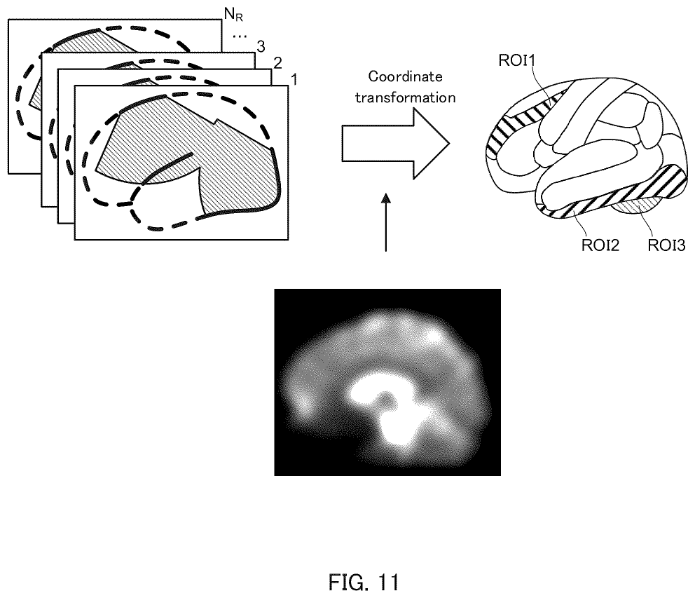

Upon acquiring the image of the entire brain in this manner, the image processor 140 may generate an image on three-dimensional atlas coordinates representing a plurality of brain regions based on the brain image constructed from the measurement results. For example, when the transmission and reception pattern for the ultrasound signals of the ultrasound probes 110_A to 100_F is repeated Nᵣ times, the image processor 140 can acquire a wide-area brain image of the entire brain from a plurality of local brain images provided by the respective ultrasound probes 110 and perform coordinate transformation on the wide-area brain image into a standard brain image prepared in advance to acquire an image of the same space as the three-dimensional brain atlas as illustrated in FIG. 11.

More specifically, upon acquiring the measurement results of the ultrasound probes 110_A to 110_F from the controller 130, the image processor 140 performs analog-to-digital (AD) conversion on signals received at the ultrasound probes 110_A to 110_F for the acquired measurement results. Then, as illustrated in FIG. 9, the image processor 140 generates the local brain images at respective ultrasound probes 110 by utilizing beamforming corresponding to the positions of the virtual sound sources for forming the spherical diffusion waves. For example, in order to uniformly grasp blood cells flowing through blood vessels with respect to the blood vessels running in various directions, the number Nₐ of virtual point sound source positions may be set in a planar space, and the propagating directions of the spherical diffusion waves may be three-dimensionally tilted Nₐ times. Backscatters may be coherently added while the images measured at Nₐ virtual point sources are averaged to reduce the angular dependency on the blood vessels running in the brain.

Then, the frame rate F for image measurement using one ultrasound probe 110 may be in accordance with Expression (1):

$$\begin{matrix}[ Number 1 ] & \\ F = \frac{c}{2 Z} \frac{1}{{N}_{a}} , & ( 1 )\end{matrix}$$

where Z is the maximum depth of the image, and c is the speed of sound (e.g., 1, 540 [m/sec]) in the living tissue.

As described above, if Nₚ ultrasound probes 110 (for example, Nₚ is an integer of 2 to 6) are sequentially driven and this sequential driving is repeated Nᵣ times, each of the ultrasound probes 110_A to 110_F may average the received signals for the emitted ultrasound signals. In this case, a frame rate F_{w} for image measurement may be in accordance with Expression (2):

$$\begin{matrix}[ Number 2 ] & \\ {F}_{w} = \frac{c}{2 Z} \frac{1}{{N}_{a}} \frac{1}{{N}_{p}} \frac{1}{{N}_{r}} , & ( 2 )\end{matrix}$$

where Nₐ is the number of virtual point sound sources. For example, if Nₐ=9, Z=5 [cm], Nₚ=6, and Nᵣ=200, the frame rate F_{w} for the image measurement using all the ultrasound probes 110_A to 110_F is 1.42 [Hz], and local brain images are acquired through all the six acoustic windows in a time period of 0.7 seconds.

With respect to the maximum frequency of 0.3 Hz included in the hemodynamics associated with neural excitation, the frame rate of 1.42 Hz is sufficiently higher than its Nyquist frequency of 0.6 Hz. It is thus possible to accurately detect cerebral hemodynamics associated with the neural excitation. However, the present disclosure is not limited thereto, and the number Nₐ of virtual point sound sources, the number Nᵣ of repetitions, and the number Nₚ of ultrasound probes may be adjusted such that the frame rate F_{w} is equal to or higher than the Nyquist frequency of 0.6 Hz regardless of the number of elements and the array of the transducers of the ultrasound probes 110 used for transmitting and receiving ultrasound waves and the driving order of the ultrasound probes 110.

To emphasize the movement of the blood cells, the image processor 140 may, for example, apply a high-pass filter to a signal value S measured using each of the ultrasound probes 110, so as to remove a clutter signal of a low-frequency component derived from the tissue. Then, the image processor 140 can average the signal values S using Expression (3) to acquire the cerebral blood flow value I by:

$$\begin{matrix}[ Number 3 ] & \\ I = \frac{1}{{N}_{r}} {\sum }_{i = 1}^{{N}_{r}} {\vert S ( {t}_{i} ) \vert}^{2} . & ( 3 )\end{matrix}$$

For the local brain image measured by each of the ultrasound probes 110, the virtual sound source coordinates and the tilted angle of the ultrasound probe 110 are known by selecting the head holder 120 used by the subject. As illustrated in FIG. 10A, the image processor 140 may arrange the local brain images in a grid coordinate space by using the nearest neighbor search to automatically superimpose the local brain images each other. For example, a voxel value having a greater average or a higher signal-to-noise ratio is employed for coordinates overlapping between the local brain images by the respective ultrasound probes 110 in the grid coordinate space. The image processor 140 can generate the wide-area brain image of the entire brain from the local brain images by superimposing the overlapping portions each other.

Note that in cases where the probe holder 121 is held by a rubber net or a band, the virtual sound source coordinates may be displaced from an intended position. For this reason, the image processor 140 may perform registration of the wide-area brain image to a standard brain template having a population-mean brain shape of infants, which is constructed by any imaging apparatus such as a Magnetic Resonance Imaging (MRI) or Computed Tomography (CT) apparatus, through rigid or non-rigid image transformation to perform coordinate transformation on the wide-area brain image to the standard brain template in the standard brain space.

For the wide-area brain image acquired by Nᵣ measurement repetitions, the image processor 140 acquires four-dimensional time-series image data 1 to N_{R} of the wide-area brain image for the measurement time Nᵣ/F_{w}, as illustrated in FIG. 11. The image processor 140 may display the four-dimensional time-series image data acquired in this manner and three-dimensional image data acquired by adding these four-dimensional time-series image data with respect to the time axis as wide-area brain morphology and a vessel and blood-flow-change image on a display or the like.

Specifically, the image processor 140 performs coordinate transformation through rigid or non-rigid registration to the standard brain template, and applies the transformation information to the cerebral blood flow image having the same coordinates as the brain morphology to transform the wide-area brain image into the standard brain coordinates. Next, the image processor 140 utilizes the three-dimensional brain atlas having the same coordinates as the standard brain coordinates to perform Region Of Interest (ROI) analysis on an ROI corresponding to each brain region for acquisition of a signal change of each ROI. Finally, in order to evaluate the functional connectivity between the brain regions, the image processor 140 applies a band-pass filter to extract components of 0.01 to 0.1 Hz in which the fluctuation in cerebral blood flow signal caused by the neural activity such as the neural excitation and perform correlation analysis between two ROIs.

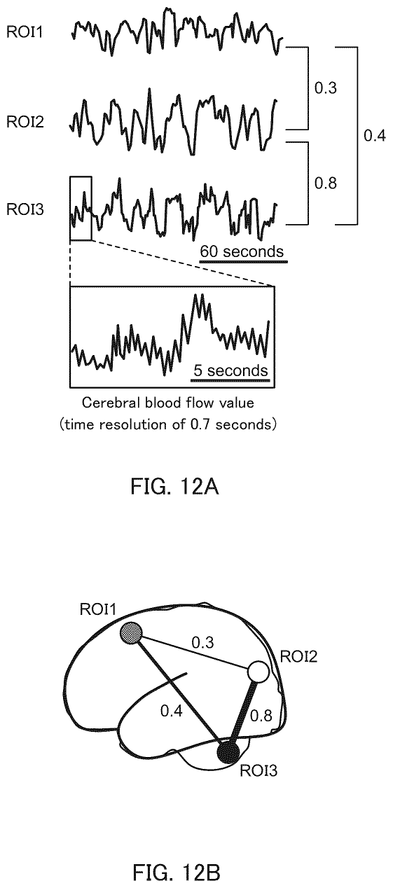

Then, the image processor 140 may present a correlation coefficient derived by the correlation analysis as the strength of connectivity between the ROIs on the standard brain space. For example, as illustrated in FIG. 12A, if the correlation coefficient between ROI1 and ROI2 is 0.3, the correlation coefficient between ROI1 and ROI3 is 0.4, and the correlation coefficient between ROI2 and ROI3 is 0.8, the image processor 140 may display a three-dimensional brain space on the display on which the brain functional network information among ROI1, ROI2, and ROI3 as illustrated in FIG. 12B is superimposed.

Here, assuming that the cerebral blood flow values of ROIᵢ and ROIⱼ at a time point tₙ are denoted by Iᵢ(tₙ) and Iⱼ(tₙ), respectively, functional connectivity FCᵢⱼ between ROIᵢ and ROIⱼ can be derived from the Pearson correlation in accordance with Expression (4):

$$\begin{matrix}[ Number 4 ] & \\ {FC}_{ij} = \frac{{\sum }_{n = 1}^{{N}_{R}} ( {I}_{i} ( {t}_{n} ) - \overline{{I}_{ι} ( t )} ) ( {I}_{j} ( {t}_{n} ) - \overline{{I}_{j} ( t )} )}{\sqrt{\frac{1}{{N}_{R}} {\sum }_{n = 1}^{{N}_{R}} {( {I}_{i} ( {t}_{n} ) - \overline{{I}_{ι} ( t )} )}^{2}} \sqrt{\frac{1}{{N}_{R}} {\sum }_{1}^{{N}_{R}} {( {I}_{n = 1} ( {t}_{n} ) - \overline{{I}_{j} ( t )} )}^{2}}} , & ( 4 )\end{matrix}$$

$$\begin{matrix}[ Number 5 ] & \\ \overline{{I}_{ι} ( t )} , \overline{{I}_{j} ( t )} & \end{matrix}$$

are average values of Iᵢ(tₙ) and Iⱼ(tₙ), respectively.

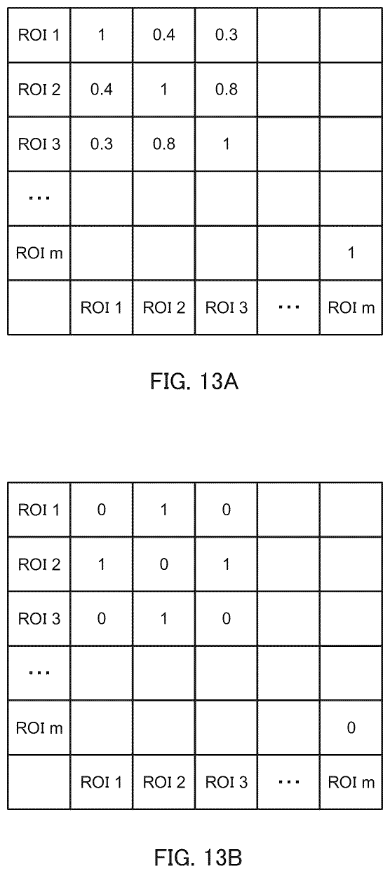

The image processor 140 generates the brain functional network information based on the functional connectivity FC between the brain regions of the entire brain acquired as described above. Specifically, for the number m of ROIs in the three-dimensional brain atlas, the image processor 140 may calculate the respective functional connectivity FC (i.e., the correlation coefficients) between the ROIs, and generate an m×m adjacency matrix with respect to the number m of ROIs in the three-dimensional brain atlas as illustrated in FIG. 13A. That is, FCᵢⱼ (=FCⱼᵢ) is input as an entry at the ith row and jth column (and the jth row and ith column) of the adjacent matrix. Further, the image processor 140 may set the diagonal components of the adjacent matrix to 0, and the network analysis may be performed on the adjacent matrix binarized by predetermined thresholds, as illustrated in FIG. 13B.

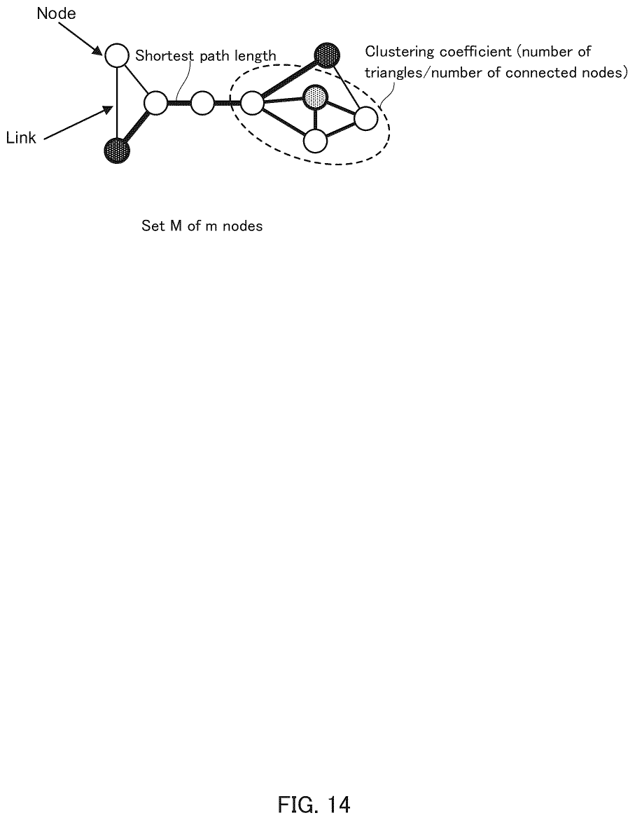

The image processor 140 may derive a functional integration index and a functional separation index of the brain in addition to the above-described brain functional network information. Specifically, for the network analysis based on FIG. 13B, the brain functional network information may be represented in a graph structure in which the ROIs are nodes and the functional connectivity FC is links as illustrated in FIG. 14. In this graph, a set M of nodes, a degree kᵢ of each node (the number of links connected to the node i), a shortest path length dᵢⱼ (the shortest distance between nodes i and j), a clustering coefficient cᵢ (a value acquired by dividing the number of triangles having the node i as one node and neighboring nodes connected to each other as nodes by the number of links connected to the node i), and the like may be used.

The degree kᵢ of the node i is provided by Expression (5) as the sum of values of the ith row among the values aᵢⱼ (0 or 1) of the ith row and the jth column of the binarized adjacent matrix A:

$$\begin{matrix}[ Number 6 ] & \\ {k}_{i} = {\sum }_{j ∈ N} {a}_{i j} . & ( 5 )\end{matrix}$$

The shortest path length dᵢⱼ is the shortest path length between two nodes i and j and is provided by Expression (6):

$$\begin{matrix}[ Number 7 ] & \\ {d}_{i j} = {\sum }_{{a}_{u ν} ∈ {g}_{i ↔ j}} {a}_{u ν} , & ( 6 )\end{matrix}$$

$$\begin{matrix}[ Number 8 ] & \\ {g}_{i ↔ j} & \end{matrix}$$

represents the shortest path between nodes i and j, and the length between nodes i and j is calculated by summing aᵤᵥ included in the shortest path. The shortest path length dᵢⱼ may be used as a measure of functional integration in the network between the brain regions.

Meanwhile, the clustering coefficient cᵢ is calculated from the degree kᵢ of the node i and the number tᵢ of triangles composed of the node i and the neighboring nodes, and can be used as a measure of the functional separation in the network between the brain regions.

$$\begin{matrix}[ Number 9 ] & \\ {t}_{i} = \frac{1}{2} {\sum }_{j , h ∈ M} {a}_{i j} {a}_{i h} {a}_{j h} & ( 7 )\end{matrix}$$

By Expressions (5) and (7), the clustering coefficient cᵢ is derived as Expression (8):

$$\begin{matrix}[ Number 10 ] & \\ {c}_{i} = \frac{{t}_{i}}{\frac{{k}_{i} ( {k}_{i} - 1 )}{2}} . & ( 8 )\end{matrix}$$

The image processor 140 may apply such a network analysis technique to wide-area brain functional activity data and display the derived network measures together with the brain functional network information on the display. This allows the user to evaluate the functional separation and functional integration in n the wide-area brain network of a subject quantitatively.

In the ultrasound device 100 illustrated in FIG. 1, a plurality of ultrasound probes 110 are provided to the head holder 120 worn on the head of a subject such as a neonate such that the ultrasound probes correspond to respective head regions. However, the present disclosure is not limited thereto, and may be realized by other configurations capable of transmitting and receiving ultrasound signals to and from the head regions of the subject.

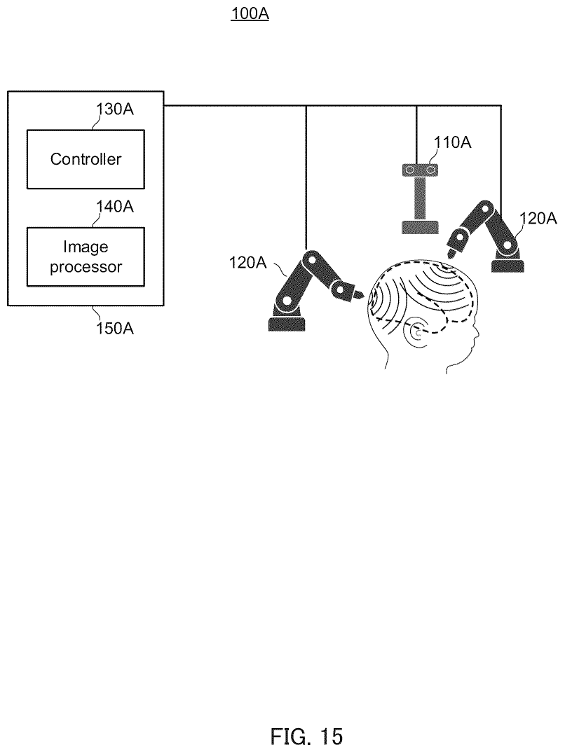

FIG. 15 schematically illustrates an ultrasound device according to another embodiment of the present disclosure. As illustrated in FIG. 15, an ultrasound device 100A includes a camera 110A, robotic arms 120A, a controller 130A, and an image processor 140A. That is, the ultrasound device 100A uses the camera 100A and the robotic arms 120A instead of the head holder 120 of the ultrasound device 100 as described above. In the illustrated exemplary embodiment, the ultrasound device 100A includes the camera 110A and the robotic arms 120A, but the present disclosure is not limited thereto. For example, the camera 110A and/or the robotic arms 120A may be utilized as external devices of the ultrasound device 100A and may be controlled by the ultrasound device 100A, for example, via a wired or wireless connection.

The camera 110A captures images of a plurality of head regions. Specifically, the camera 110A may be realized by a stereo camera or the like, and may capture an image of the head of the subject to acquire 4D measurement data on a head surface shape of the subject. Further, for example, the camera 110A may be capable of controlling an optical system such as a lens to follow the plurality of head regions in response to the movement of the subject under the control of the controller 130A.

The robotic arms 120A include an ultrasound probe 121A at a distal end or the like of the arm, and the ultrasound probe 121A transmits an ultrasound signal toward an object and receives a reflected signal from the object, similar to the ultrasound probe 110 described above. Under the control of the controller 130A, the robotic arm 120A identifies and tracks a plurality of head regions of the subject based on images of the head of the subject captured by the camera 100A, transmits ultrasound signals toward the plurality of head regions of the subject by the ultrasound probe 121A, and receives reflected signals from these head regions. Typically, a plurality of robotic arms 120A may be included in the ultrasound device 100A to transmit and receive the ultrasound signals to and from the plurality of head regions.

The controller 130A controls the camera 110A and the robotic arms 120A. Specifically, the controller 130A controls the movement of the robotic arms 120A based on the images of the head regions of the subject captured by the camera 110A such that the ultrasound signals can be transmitted and received between to-be-examined head regions and the ultrasound probes 121A. For example, the controller 130A may control movement of the robotic arms 120A such that the ultrasound probes 121A can follow the head regions during transmission and reception of ultrasound signals. Upon acquiring measurement results from the ultrasound probes 121A of the plurality of robotic arms 120A, the controller 130A passes the acquired measurement results to the image processor 140A. The image processor 140A performs the same image processing as that of the above-described image processor 140 on the acquired measurement results to generate brain functional network information.

For example, the robotic arms 120A operate based on the three-dimensional shape of an infant's head acquired from the camera 110A. To begin with, the disposed positions of the ultrasound probes on the head and the angles of application of the ultrasound probes may be estimated from the acquired three-dimensional head shape by image processing and/or point cloud data processing. The controller 130 controls the robotic arms 120A based on the estimated position/angle information such that the ultrasound probes 121A are disposed in predetermined head regions. Further, the controller 130A may control the positions and the angles of the robotic arms 120A such that the robotic arms can track the movement of the head of the infant using the three-dimensional information acquired from the camera 110A in real time and the relative positional relation between the ultrasound probes 121A and the head is always the same. Note that the camera 110A may be any other cameras as long as it can acquire the three-dimensional shape information in real time. The robotic arms 120A having the ultrasound probes 121A mounted may not only track the movement of the head by the camera 110A but also automatically adjust the positions and angles of the ultrasound probes 121A to maximize the field of view and the contrast of the brain image data of the infant measured by the ultrasound probes 121A.

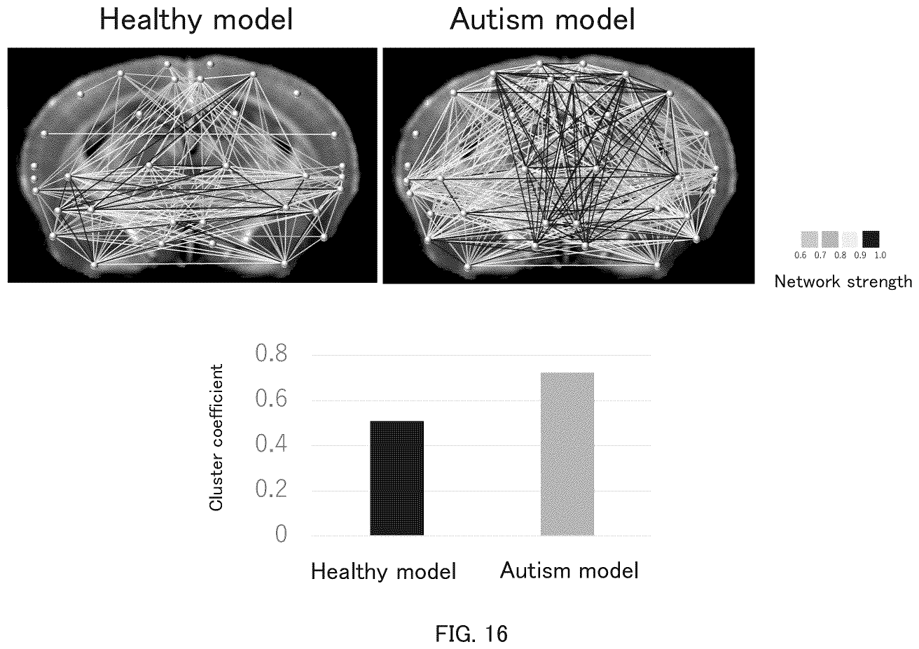

One of the developmental disorders, autism spectrum disorder, is mainly characterized by deficits in communication, language impairment, and repetitive behaviors (see the diagnostic criteria of the American Psychiatric Association; DSM-V, 2013). A study using MRI shows that strong local connectivity exists as a feature in cerebral networks of the autism spectrum disorder (Belmonte, J Neurosci 2004). A maternal immune activation model used in preclinical research for the developmental disorders is widely used as autism phenotypes of social deficits and increase in repetitive behaviors (Choi, Science 2016) in research for elucidating the condition of autism. Brain network measurement using ultrasound waves on this autism model observed an excessive brain network in a wide area of a brain including a deep part of the brain as in autistic patients. The network analysis of such a brain network showed the result that the clustering coefficient for evaluating the local connectivity is greater than that of a healthy model (FIG. 16). Therefore, it is conceivable that features of a brain network of an infant in a developmental disorder are applied to the examination method using the network index in one exemplary embodiment of the present disclosure for evaluating the wide-area brain network by using ultrasound waves.

### [Ultrasound Signal Processing]

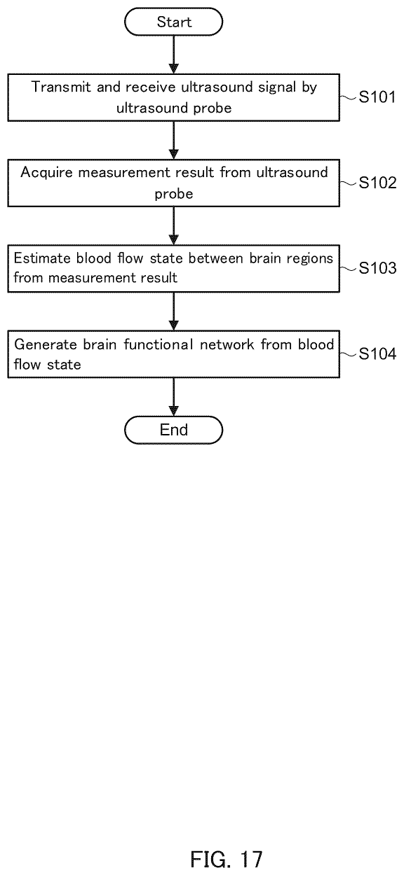

Referring now to FIG. 17, ultrasound signal processing according to one exemplary embodiment of the present disclosure is described. The ultrasound signal processing may be performed by the above-described ultrasound device 100, and more particularly, implemented by one or more processors of the ultrasound device 100 executing one or more programs or instructions stored in one or more memories.

FIG. 17 is a flowchart illustrating the ultrasound signal processing according to one exemplary embodiment of the present disclosure.

As illustrated in FIG. 17, in step S101, the ultrasound device 100 uses the ultrasound probes 110 to transmit and receive ultrasound signals to and from brain regions in the brain through acoustic windows in the head of a subject. In the present exemplary embodiment, the ultrasound probes 110 are mounted on the head holder 120 corresponding to the plurality of head regions of the infant. For example, the head regions used as the acoustic windows for the ultrasound probes 110 may include two or more of the anterior fontanelle, the posterior fontanelle, the sphenoidal fontanelle, and the mastoid fontanelle that open in the head of the infant. The head holder 120 is provided with the probe holders 121 for holding the respective ultrasound probes 110 such that the ultrasound probes 110 are placed in these head regions when the infant wears the head holder 120. The ultrasound probes 110 transmit the ultrasound signals to the brain of the subject through the acoustic windows serving as openings at which the ultrasound probes are placed. Then, the ultrasound probes receive the reflected ultrasound signals. The ultrasound device 100 may control the transmission and reception pattern for the ultrasound signals by the ultrasound probes 110, for example, by driving the plurality of ultrasound probes 110 one after another. Based on differences in the transmitted and received ultrasound signals between a transmission timing and a reception timing, the ultrasound probes 110 can acquire measurement results regarding the respective brain regions in the vicinity of the openings.

In step S102, the ultrasound device 100 acquires the measurement results from the ultrasound probes 110. Specifically, the ultrasound device 100 acquires the measurement results for the brain regions in the vicinity of the placement positions of the ultrasound probes from the ultrasound probes 110 and generates local brain images based on the measurement results from the ultrasound probes 110. Each of the local brain images shows the blood flow state of one or more brain regions.

In step S103, the ultrasound device 100 estimates the blood flow state among a plurality of brain regions in the brain from the measurement results. Specifically, the ultrasound device 100 may identify overlapping portions between the local brain images acquired from each of the ultrasound probes 110 and the local brain image acquired from an adjacent ultrasound probe 110 and combine the adjacent local brain images by superimposing the identified overlapping portions each other. The ultrasound device 100 repeats combination of the local brain images based on the overlapping portions for all the local brain images and generates a wide-area brain image of the entire brain. Then, the ultrasound device 100 estimates a change caused in the blood flow state in each brain region as a hemodynamic response to neural excitation and performs correlation analysis on the blood flow state between the brain regions. For example, if the correlation coefficient calculated for the brain regions A and B is equal to or greater than a predetermined threshold, it may be determined that the brain regions A and B have functional connectivity. On the other hand, if the calculated correlation coefficient for the brain regions A and B is less than the predetermined threshold, it may be determined that the brain regions A and B do not have functional connectivity.

In step S104, the ultrasound device 100 may determine the presence or absence of the above-described functional connectivity for each pair of brain regions to acquire the brain functional network information on a brain functional network between the brain regions as a graph structure based on determination results.

According to the above-described exemplary embodiment, cerebral hemodynamics associated with neural excitation are observed as in the brain functional magnetic resonance imaging. It is thus possible to perform brain-activity and brain-functional network analyses. In addition, it is possible to provide infants with a wide range of approaches for functional network evaluation in which a wide brain area is grasped integrally. Accordingly, it is possible to expect a wide range of applications to early diagnosis of developmental disorders suspected as being brain-activity or brain-functional network abnormality, understanding of the brain in the developmental stage, and a neuromarketing industry such as development of toys for infants and children, and other applications.

Although the exemplary embodiments of the present disclosure have been described in detail above, the present disclosure is not limited to the specific embodiments described above, and various modifications and variations can be made within the scope of the gist of the present disclosure described in the claims.

Further, the following additional notes are disclosed with respect to the above description.

### (Additional Note 1)

An ultrasound device, including:
a plurality of ultrasound probes that transmit and receive an ultrasound wave to and from a plurality of brain regions via a plurality of head regions, the plurality of ultrasound probes being disposed to correspond to the respective head regions;
a controller that controls the plurality of ultrasound probes; and
an image processor that generates brain functional network information calculated from a blood flow state among the plurality of brain regions based on a measurement result acquired from the plurality of ultrasound probes.

### (Additional Note 2)

The ultrasound device according to additional note 1, further including: a head holder provided with the plurality of ultrasound probes.

### (Additional Note 3)

The ultrasound device according to additional note 1, further including:
a camera that captures the plurality of head regions; and
a plurality of robotic arms provided with the plurality of ultrasound probes.

### (Additional Note 4)

The ultrasound device according to additional note 3, wherein the controller controls movement of the plurality of robotic arms based on captured images of the plurality of head regions such that the plurality of robotic arms follow the plurality of head regions.

### (Additional Note 5)

The ultrasound device according to any one of additional notes 1 to 4, in which the plurality of head regions include two or more of an anterior fontanelle, a posterior fontanelle, a left sphenoidal fontanelle, a right sphenoidal fontanelle, a left mastoid fontanelle, a right mastoid fontanelle, and a temporal bone window.

### (Additional Note 6)

The ultrasound device according to any one of additional notes 1 to 5, in which the controller controls the plurality of ultrasound probes such that the plurality of ultrasound probes transmit and receive ultrasound waves alternately.

### (Additional Note 7)

The ultrasound device according to any one of additional notes 1 to 6, in which each of the plurality of ultrasound probes has a mechanism for forming a wavefront of an arbitrary shape, and transmits the ultrasound wave using the mechanism.

### (Additional Note 8)

The ultrasound device according to any one of additional notes 1 to 7, in which the image processor generates an image representing the plurality of brain regions on a three-dimensional atlas coordinate based on a brain image constructed from the measurement result.

### (Additional Note 9)

The ultrasound device according to any one of additional notes 1 to 8, in which the image processor generates the brain functional network information based on a correlation of a temporal change of a cerebral blood flow signal intensity among the plurality of brain regions.

### (Additional Note 10)

The ultrasound device according to any one of additional notes 1 to 9, in which the brain functional network information is represented as a graph structure.

### (Additional Note 11)

A head holder, including:
a plurality of ultrasound probes that transmit and receive an ultrasound wave to and from a plurality of brain regions via a plurality of head regions,
wherein the plurality of ultrasound probes are disposed corresponding to the respective head regions.

### (Additional Note 12)

An ultrasound signal processing method executed by a computer, the ultrasound signal processing method including:
acquiring a measurement result from a plurality of ultrasound probes that transmit and receive ultrasound waves to and from a plurality of brain regions via a plurality of head regions; and
generating brain functional network information calculated from a blood flow state among the plurality of brain regions based on the measurement result,
wherein the plurality of ultrasound probes are disposed corresponding to the respective head regions.

### REFERENCE SIGNS LIST

100, 100A Ultrasound device
110, 121A Ultrasound probe
110A Camera
120 Head holder
120A Robotic arm
121 Probe holder
122 Silicon pad
123 Ultrasound gel
130, 130A Controller
140, 140A Image processor
150 Control device

1. An ultrasound device, comprising:
a plurality of ultrasound probes that transmit and receive an ultrasound wave to and from a plurality of brain regions via a plurality of head regions, the plurality of ultrasound probes being disposed corresponding to the respective head regions;
a controller that controls the plurality of ultrasound probes; and
an image processor that generates brain functional network information calculated from a blood flow state among the plurality of brain regions based on a measurement result acquired from the plurality of ultrasound probes.

2. The ultrasound device according to claim 1, further comprising:
a head holder provided with the plurality of ultrasound probes.

3. The ultrasound device according to claim 1, further comprising:
a camera that captures the plurality of head regions; and
a plurality of robotic arms provided with the plurality of ultrasound probes.

4. The ultrasound device according to claim 3, wherein the controller controls movement of the plurality of robotic arms based on captured images of the plurality of head regions such that the plurality of robotic arms follow the plurality of head regions.

5. The ultrasound device according to claim 1, wherein the plurality of head regions include two or more of an anterior fontanelle, a posterior fontanelle, a left sphenoidal fontanelle, a right sphenoidal fontanelle, a left mastoid fontanelle, a right mastoid fontanelle, and a temporal bone window.

6. The ultrasound device according to claim 1, wherein the controller controls the plurality of ultrasound probes such that the plurality of ultrasound probes transmit and receive ultrasound waves alternately.

7. The ultrasound device according to claim 1, wherein each of the plurality of ultrasound probes has a mechanism for forming a wavefront of an arbitrary shape, and transmits the ultrasound wave using the mechanism.

8. The ultrasound device according to claim 1, wherein the image processor generates an image representing the plurality of brain regions on a three-dimensional atlas coordinate based on a brain image constructed from the measurement result.

9. The ultrasound device according to claim 1, wherein the image processor generates the brain functional network information based on a correlation of a temporal change of a cerebral blood flow signal intensity among the plurality of brain regions.

10. The ultrasound device according to claim 1, wherein the brain functional network information is represented as a graph structure.

11. A head holder, comprising:
a plurality of ultrasound probes that transmit and receive an ultrasound wave to and from a plurality of brain regions via a plurality of head regions,
wherein the plurality of ultrasound probes are disposed corresponding to the respective head regions.

12. An ultrasound signal processing method executed by a computer, the ultrasound signal processing method comprising:
acquiring a measurement result from a plurality of ultrasound probes that transmit and receive ultrasound waves to and from a plurality of brain regions via a plurality of head regions; and
generating brain functional network information calculated from a blood flow state among the plurality of brain regions based on the measurement result,
wherein the plurality of ultrasound probes are disposed corresponding to the respective head regions.
